(19) United States
(12) Patent Application Publication
(10) Pub. No.: US 2006/0253081 A1
(43) Pub. Date: Nov. 9, 2006

(54) Cannulated injection system

(76) Inventors: Lonnie E. Paulos, Salt Lake City, UT (US); Robert A. Van Wyk, Largo, FL (US)

Correspondence Address: Lonnie E. Paulos;Suite 200, 5250 So. Commerce Drive, Salt Lake City, UT, 84107, US

(21) Appl. No.: 11/112,550

(22) Filed: Apr. 25, 2005

(51) Int. Cl. A61M 5/00

(52) U.S. Cl. 604/187

(57) ABSTRACT

A cannulated injection system having or more hollow bodies to receive material to be injected, a piston in each said hollow body to force material from said body through a nozzle having a passageway sized and selected to accommodate flow of the material therethrough and with the passageway arranged to accommodate a guide wire passed therethrough, the guide wire further being passed through support structure including a piston and hollow body in which said piston reciprocates or a support structure for a plurality of hollow bodies in which pistons reciprocates during use in simultaneously injecting material from said hollow bodies through said injection tip and around a guide wire passing through the tip.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO MICROFICHE APPENDIX

Not Applicable.

### BACKGROUND OF THE INVENTION

1. Field of the Invention

This invention relates to injection systems and more particularly, to a cannulated injection system for orthopedic applications which may be guided to its desired location by a previously placed guide wire.

2. Description of the Prior Art

Recently a number of healing enhancing products have come to market. These include calcium triphosphates, high hyaluronic acid products, platelet healing factors and others. These materials are used to enhance bone tunnel fixation of soft tissue ligament grafts as well as arthroscopically treating non-unions of long bones, bone cysts of the hip, knee and other related skeletal abnormalities. The sites at which the healing enhancing products must be placed are frequently difficult to access making the required accurate placement of the materials difficult. This is particularly true for endoscopic techniques. The injection system must be well suited to the site and material placement requirements of the procedure. Sites more remote from the access point require a device with an elongated delivery nozzle. Additionally, some applications require a large diameter nozzle for uniform material distribution while others require a smaller diameter nozzle for precise material placement.

The use of guide wires in orthopedic surgery, particularly arthroscopy, is common. A guide wire (a long, rigid, small diameter, stainless steel member), or a small diameter flexible stainless steel cable is precisely positioned at the desired site and its position verified by direct visualization. The guide wire or cable is then inserted into the cannulation of a cannulated instrument and the instrument is precisely guided to the desired site by the wire or cable. Guide wires and cables are produced in a range of diameters and lengths to suit a variety of procedures.

The viscosity of the materials to be injected also affects the choice of an injection device and the manner of use of such device. The materials may include liquified products, emulsified products or even slurries and, as such, may have quite high viscosities. Liquids are commonly drawn into a syringe through the needle. The distal end of the syringe is then directed upward and the piston advanced to expel air from the syringe. To load higher viscosity materials into a syringe the piston must be removed and the materials loaded into the body of the syringe through its proximal end. The material settles into the distal end of the syringe body. When the piston is inserted into the proximal end of the syringe body, air is trapped between the piston distal end and the material. Advancing the piston will cause material to be expelled from the device. With currently available devices, removing trapped air is problematic when materials are loaded through the proximal end of the syringe body.

U.S. Pat. No. 6,395,007 discloses apparatus and method for the fixation of osteoporotic bone. The patent discloses an injection device including a delivery cannular, a liner acting as an injection material conduit, a plunger capable of passing through the liner to force injectable material through the liner while permitting air to escape past the plunger and a removable handle attachable to the delivery cannulae.

Further, the patent discloses use of a guide wire passed through an aligning cannulae and having a tapered end that will breach cortical bone sufficient to form a channel through the cortical bone. An aligning cannulae passes over the guide wire and with the delivery cannulae passing over the aligning cannulae. The injectable materials disclosed include polymethylmetaery, bone cement, antibiotics, whole cellular implants, natural products of cells, recombinant nucleic products and protein products of recombinant cells.

### SUMMARY OF THE INVENTION

### Objects of the Invention

It is an object of this invention to produce an injection system which may be guided to its desired position by a guide wire or cable.

It is also an object of this invention to produce an injection system which may be guided to its desired position by a guide wire and which may accommodate guide wires or cables having a range of diameters.

It is also an object of this invention to produce an injection system which may be guided to its desired position by a guide wire and which has a range of nozzle diameters and lengths suitable for a variety of applications.

It is further an object of this invention to produce an injection system which may be guided to its desired position by a guide wire and which can be used with materials, including liquefied products, emulsified products and slurries.

It is also an object of this invention to produce an injection system which may be guided to its desired position by a guide wire and from the body of which air can be expelled with the syringe distal end pointed downward.

Additional objects and features of the invention will become apparent to persons skilled in the art to which the invention pertains from the following detailed description and claims.

### Features of the Invention

Principal features of the invention herein disclosed include a cannulated injection system comprising a cannulated syringe and demountable nozzle, which may be configured for a variety of applications. A cannulated syringe has a clear hollow cylindrical body, including a cannulated distal end with a mounting means to which a nozzle can be mounted and an open proximal end into which a piston assembly is inserted. The piston assembly has a proximal end and a distal end and comprises an outer member and an inner member, with the inner member concentrically and rotatably positioned within the outer member. The inner member may be rotated by a proximal end means so that passages in the inner member and outer member distal ends align to provide an aspiration path through the piston assembly. The piston inner member comprises a cannulation of sufficient size to allow passage of common guide wires of various sizes. A seal made from silicone, or a similar material, within the inner member cannulation, prevents leakage of material through the inner member cannulation during use. A seal prevents leakage through the clearance between the piston assembly and the hollow cylindrical body.

During use a guide wire is placed to aid in positioning the syringe. The piston assembly is removed from the syringe and the desired material loaded into the hollow cylindrical body through its proximal opening. The piston inner member is rotated to an “aspirate” position. While blocking the distal end of the body to prevent loss of material, the piston assembly is inserted into the body and advanced until the piston assembly distal end contacts the upper surface of the material and all trapped air is aspirated. The piston inner member is rotated to a “ready” position. An appropriate nozzle is selected and mounted to the syringe. The syringe, with the nozzle thereon, is positioned and advanced such that the guide wire passes through the cannulation of the nozzle; through the cannulation of the inner member; through the seal, which elastically deforms to accept the guide wire diameter; and exits the piston proximal end. The syringe is advanced along the guide wire until properly positioned at a desired site. The piston is advanced in the syringe so as to deposit the desired amount of material at the site.

In an alternate embodiment for use with low-viscosity materials only, the piston assembly does not contain a means for aspirating trapped air therethrough since trapped air can be expelled by pointing the nozzle upward and advancing the piston into the body. The cannulated piston assembly does, however, contain a sealing means for preventing material loss around the guide wire and through the cannulation during use.

Some injectable, healing enhancing products are supplied as two components which are mixed immediately prior to use, the ratio of the mix being specified by the manufacturer. In another embodiment for use with these low-viscosity, two-component systems, the body of the syringe has to cylindrical barrels, the ratio of their cross-sectional areas being equal to the required mixing ratio of the components. The body of the syringe has a cannulation to allow passage of a guide wire therethrough and a sealing means to prevent material loss through the cannulation during use. The piston assembly has two pistons corresponding in size to the cylindrical barrels, the pistons being connected to a common flange at their proximal ends so that their axial movements are simultaneous.

The more important features of the invention have been outlined rather broadly in order that the detailed description thereof that follows may be better understood, and in order that the present contribution to the art may be better appreciated. There are, of course, additional features of the invention that will be described hereinafter and which will form the subject matter of the claims appended hereto.

### BRIEF DESCRIPTION OF THE DRAWINGS

In the Drawings

FIG. 1 is a front elevation view of a cannulated injection system of the invention;

FIG. 2, a side elevational view of the system of FIG. 1;

FIG. 3, a proximal end view of the system of FIG. 1;

FIG. 4, a perspective view of the system of FIG. 1;

FIG. 5, is a front elevational view of the syringe body of the invention;

FIG. 6, a side elevational view of the syringe body of FIG. 5;

FIG. 7, a proximal end view of the syringe of FIG. 5;

FIG. 8, a distal end view of the syringe body of FIG. 5;

FIG. 9, a perspective view of the syringe body of FIG. 5;

FIG. 10, is a front elevational view of the syringe piston outer member;

FIG. 11, a side elevational view of the outer member of FIG. 10;

FIG. 12, a proximal end view of the outer member of FIG. 10;

FIG. 13, a distal end view of the outer member of FIG. 10;

FIG. 14, a perspective view of the outer member of FIG. 10;

FIG. 15, is a front elevational view of the piston inner member of the invention;

FIG. 16, a side elevational view of the inner member of FIG. 15;

FIG. 17, a proximal end view of the inner member of FIG. 15;

FIG. 18, a distal end view of the inner member of FIG. 15;

FIG. 19, a perspective view of the inner member of FIG. 15;

FIG. 20, is a plan view of the piston assembly rotatable cap;

FIG. 21, a front elevational view of the rotatable cap of FIG. 20;

FIG. 22, a side elevational view of the rotatable cap of FIG. 20;

FIG. 23, a bottom view of the rotatable cap of FIG. 20;

FIG. 24, a perspective view of the rotatable cap of FIG. 20;

FIG. 25, is a plan view of the inner member retainer of the invention;

FIG. 26, a side elevational view of the retainer of FIG. 25;

FIG. 27, a perspective view of the retainer of FIG. 25;

FIG. 28, is a distal end view of the inner member seal retainer of the invention;

FIG. 29, a side elevational view of the seal retainer of FIG. 28;

FIG. 30, a perspective view of the seal retainer of FIG. 28;

FIG. 31, is a plan view of the inner member seal of the invention;

FIG. 32, a side elevational view of the seal of FIG. 31;

FIG. 33, a perspective view of the seal of FIG. 31;

FIG. 34, is a front elevational view of the piston assembly with the inner assembly rotated to its “aspirate” position;

FIG. 35, a side elevational view of the piston assembly of FIG. 34;

FIG. 36, a proximal end view of the piston assembly of FIG. 34;

FIG. 37, a distal end view of the piston assembly of FIG. 34;

FIG. 38, a perspective view of the piston assembly of FIG. 34;

FIG. 39, an expanded view of the distal portion of FIG. 34;

FIG. 40, an expanded perspective view of the distal portion of FIG. 34;

FIG. 41, is a front elevational view of the piston assembly with the inner assembly rotated to its “use” position;

FIG. 42, a side elevation view of the distal portion of FIG. 40;

FIG. 43, a proximal end view of the distal portion of FIG. 40;

FIG. 44, a distal end view of the distal portion of FIG. 40;

FIG. 45, a perspective view of the distal portion of FIG. 40;

FIG. 46, an expanded view of the distal portion of FIG. 40;

FIG. 47 is a front elevational view of the nozzle;

FIG. 48, a side elevational view of the nozzle of FIG. 47;

FIG. 49, a proximal end view of the nozzle of FIG. 47;

FIG. 50, a distal end view of the nozzle of FIG. 47;

FIG. 51, a perspective view of the nozzle of FIG. 47;

FIG. 52, is a plan view of an alternate embodiment of a cannulated injection system of the invention;

FIG. 53, a front elevational view of the system of FIG. 52;

FIG. 54, a perspective view of the system of FIG. 52;

FIG. 55, an expanded front elevational view of the piston assembly of the system of FIG. 52, showing internal features;

FIG. 56, an expanded front elevational view of the body and nozzle of the object of FIG. 52, showing internal features;

FIG. 57, is a plan view of another alternate embodiment of cannulated injection system of the invention;

FIG. 58, a front elevational view of the system of FIG. 56;

FIG. 59, a front perspective view of the system of FIG. 56;

FIG. 60, a front elevational view of the body and nozzle of the system of FIG. 57, showing internal features;

FIG. 61, a plan view of the system of FIG. 61; and

FIG. 62, an expanded perspective view of the piston assembly of the system of FIG. 57.

### DETAILED DESCRIPTION

Referring Now to the Drawings

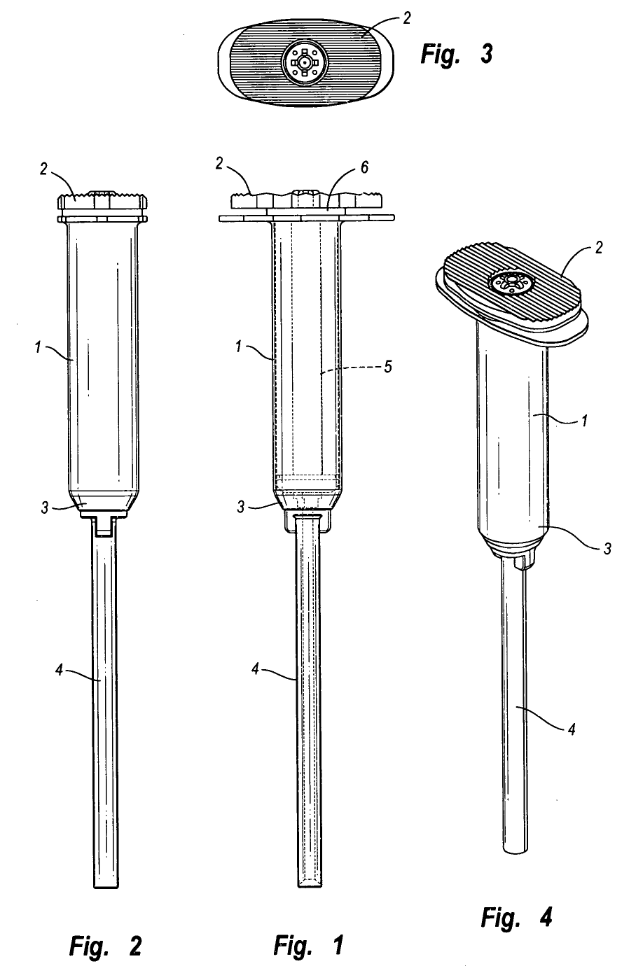

In the portion of the invention shown in FIGS. 1 through 4, cannulated injection system 1 has a proximal end 2 and a distal end 3 to which is mounted nozzle 4. System 1 has a hollow body 5 and a piston assembly 6 in the body.

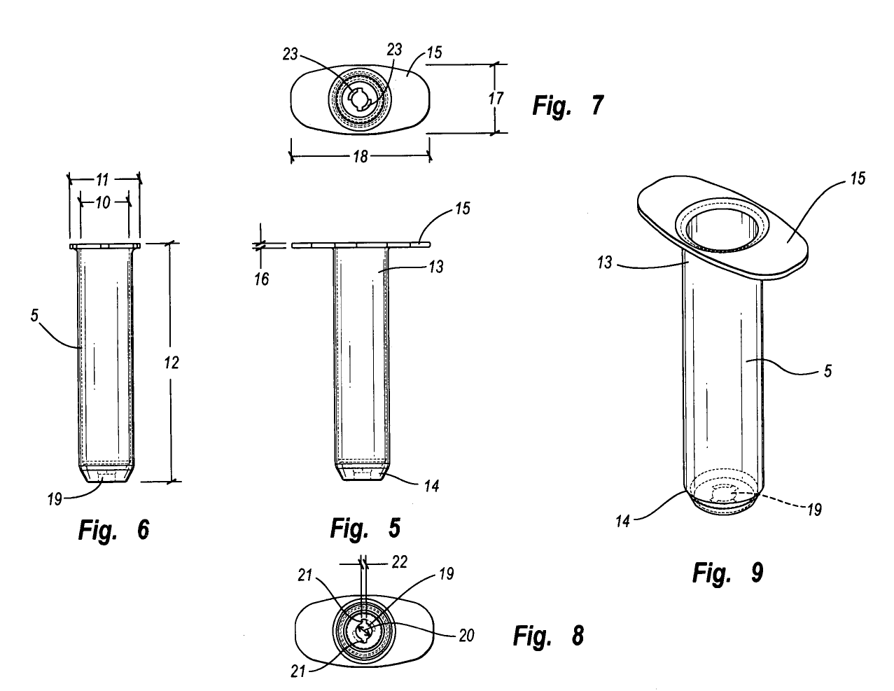

Referring to FIGS. 5-9, body 5 of inner diameter 10, outer diameter 11 and length 12 has a proximal end 13 and a distal end 14. Proximal end 13 has a flange 15 of thickness 16 and, as viewed in FIG. 7, an approximately rectangular shape of width 17 and length 18. Distal end 14 has a cannulation 19 of diameter 20, grooves 21 of width 22 and shoulders 23.

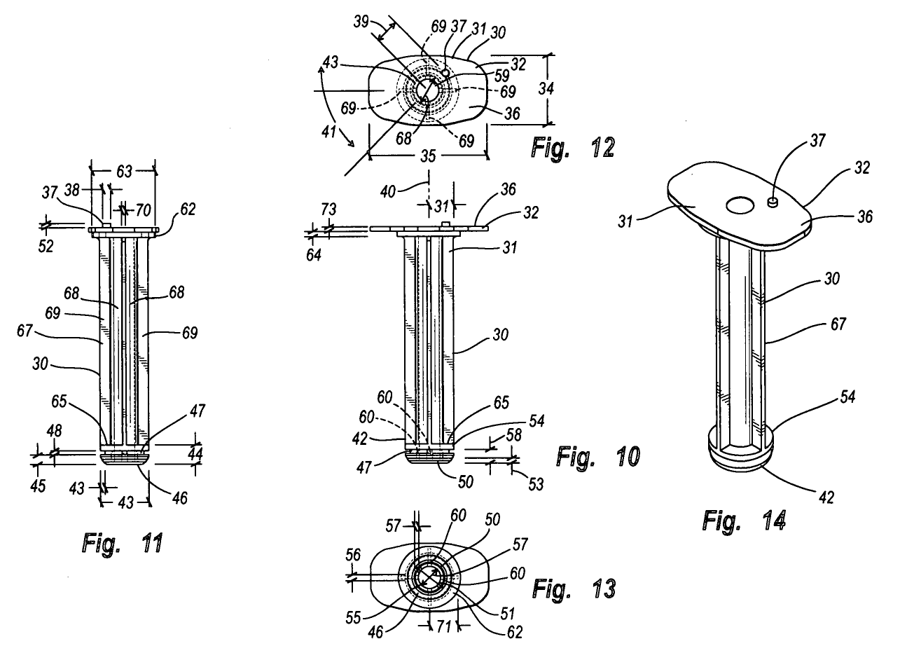

Referring to FIGS. 10-14, piston outer member 30 has a proximal end 31 having a flange 32 of thickness 33 and approximate rectangular shape of width 34 and length 35. Protruding from the most proximal surface 36 of flange 32 is cylindrical projection 37 of diameter 38 and height 52, displaced a distance 39 from axis 40 of outer member 30 and at angle 41. Distal end 42 has a cylindrical portion 54 of diameter 43 and length 44. Positioned distance 45 from the most distal surface 46 is an O-ring groove 47 of width 48 and depth 49. Centered with surface 46 is circular pocket 50 of diameter 51 and depth 53. A cannulation 59 of diameter 55 extending coaxial with axis 40 and from the most proximal surface 36 to pocket 50. Four equally spaced grooves 60 of width 56 and depth 57 extend distance 58 from a planar surface 59 of pocket 50. Distal to and adjacent to flange 32, cylindrical segment 62 of diameter 63 extends a distance 64. Coaxial with cannulation 59 and extending from most distal surface 65 of cylindrical segment 62 to most proximate surface 66 of distal cylindrical portion 54. Outer member mid-portion 67 has a circular cross-section radius 68. Four ribs 69 each have a thickness 70 and length 71 that are twice the sum of radius 68. Length 71 and diameter 43 are each slightly less than inner diameter 10 of body 5.

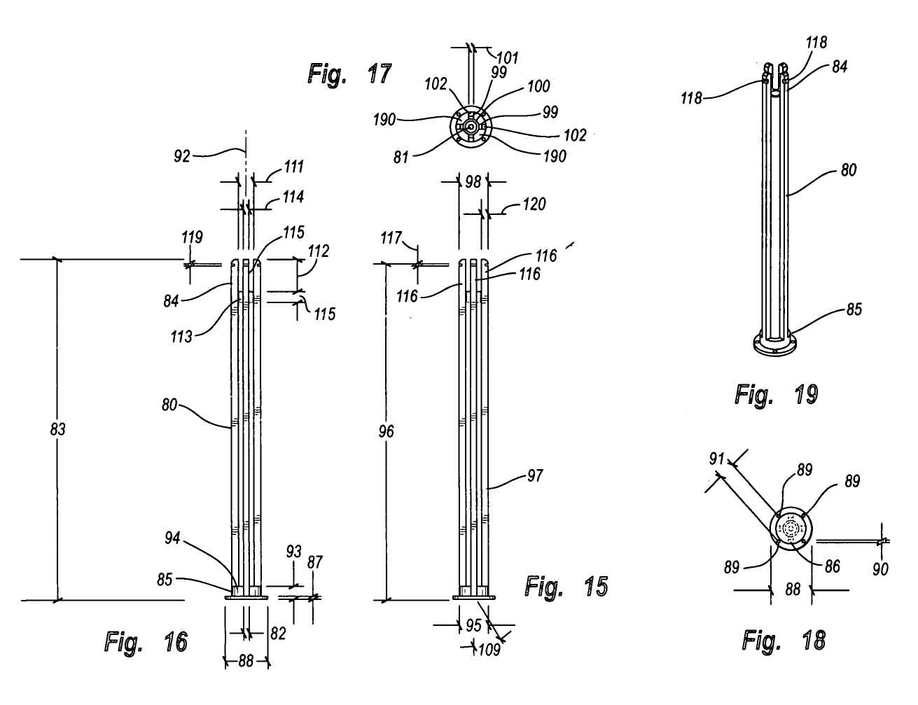

Referring to FIGS. 15-19, piston inner member 80 having a central cannulation 81 of diameter 82, has a length 83, a proximal end 84 and a distal end 85. Distal end 85 has a distal flange 86 of thickness 87 and diameter 88 that is coaxial with cannulation 81. Thickness 87 is equal to depth 53 of pocket 50 of piston outer member 30 (FIGS. 10-13) and diameter 88 is slightly less than diameter 51 of pocket 50 of piston outer member 30 (FIGS. 10-13). Four axial holes 89 of diameter 90 are equally spaced angularly in flange 86 a distance 91 from axis 92 of cannulation 81. Coaxial with flange 86 and extending distance 93 therefrom is cylindrical segment 94 of diameter 95. Diameter 95 is slightly less than the diameter 58 of grooves 60 of outer member 30 (FIGS. 10-13). Coaxial with cannulation 81 and extending proximally from cylindrical segment 94 distance 96 cylindrical segment 97 of diameter 98 has four axially oriented pockets 191, each having orthogonal planar faces 99 and radial surfaces 100 coaxial with cannulation 81 extending the length of segment 97 so as to form ribs 102 of thickness 101. Diameter 98 is slightly less than diameter 55 of cannulation 59 of outer member 30. Cylindrical pocket 110 of diameter 111 equal to twice the radius of radial faces 100 extends length 112 from distal end 84 so as to form four protruding ribs 116 of axial length 112. Coaxial with pocket 110 is cylindrical pocket 113 of diameter 114 and length 115, diameter 114 being less than diameter 111. Distance 117 from proximal end 84 is circumferential groove 118 of width 119 and depth 120. Cannulation 81 is flared at angle 109 at its distal end.

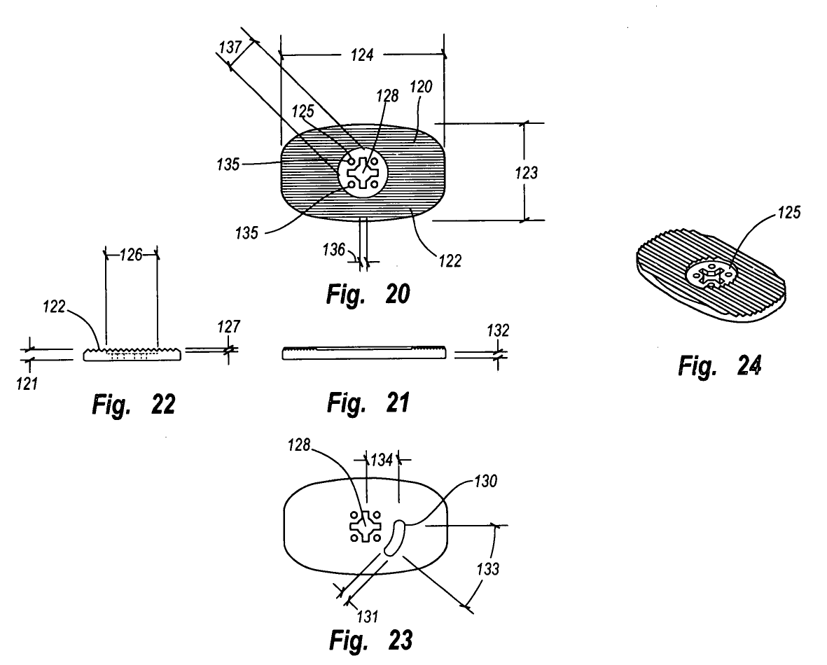

Referring to FIGS. 20-24, rotational cap 120 of thickness 121 has a serrated upper surface 122 of width 123 and length 124 equal to width 34 and length 35 of flange 32 of outer member 30. A circular recess 125 of diameter 126 and depth 127 is centered in upper surface 122. Coaxial with recess 125, passage 128 has a cross-section identical to the cross-section of segment 97 of inner member 80 except slightly larger. Radial passage 130 of width 131, depth 132 and angular length 133 is centered distance 134 from the center of cap 120, width 131 being slightly greater than diameter 38 of protrusion 37 and depth 132 being greater than length 52 of protrusion 37, and distance 134 being equal to distance 39 of outer member 30. Four holes 135 of diameter 136 are equally spaced on a circle of diameter 137 coaxial with recess 125.

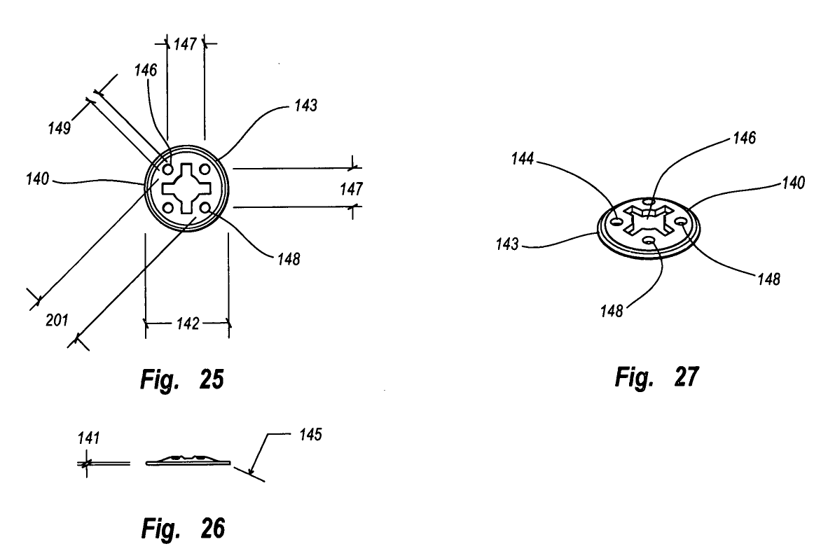

Referring to FIGS. 25-27, retainer 140 is formed from resilient stainless steel sheet material of thickness 141. Retainer 140 of diameter 142 has a perimetral planar region 143 and a central domed region 144 formed to a spherical radius 145. Central in retainer 140, opening 146 has a cross-section identical to that of opening 128 in rotational cap 120 except that distance 147 is less than diameter 98 of segment 97 of inner member 80. Four holes 148 of diameter 149 are equally spaced on a circle of diameter 201.

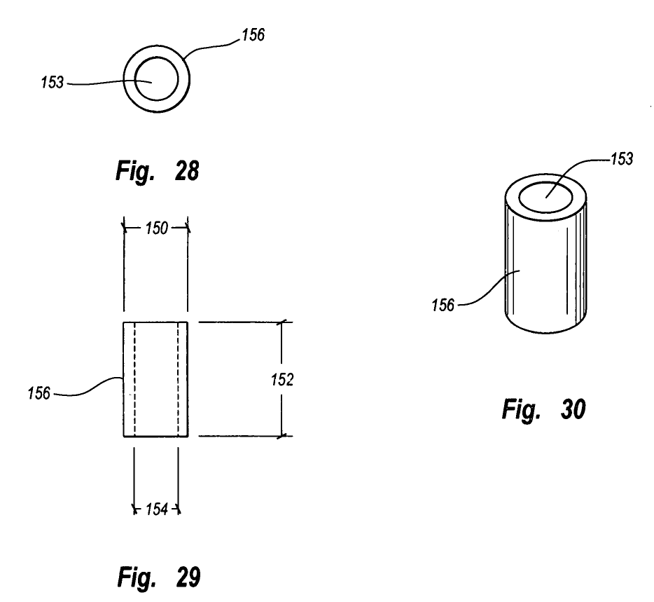

Referring to FIGS. 28-30, seal retainer 150 has outer diameter 151, length 152 and cannulation 153 of diameter 154 and is made of a rigid polymeric material.

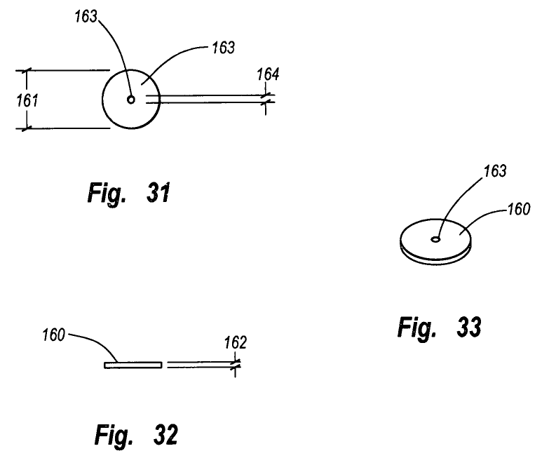

Referring to FIGS. 31-33, seal 160 of diameter 161 and thickness 162 is made from a compliant silicone material or similar. Centered in seal 160 is hole 163 of diameter 164.

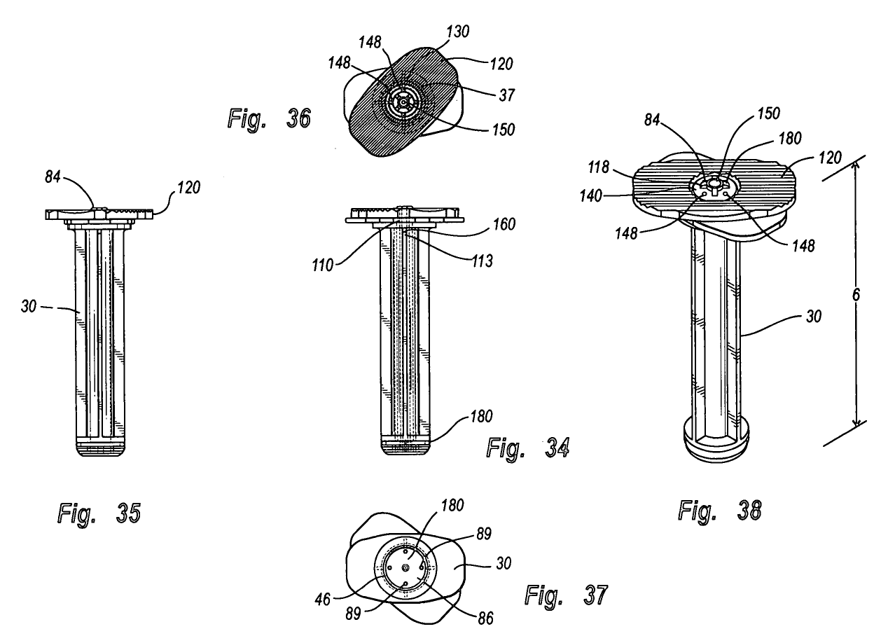

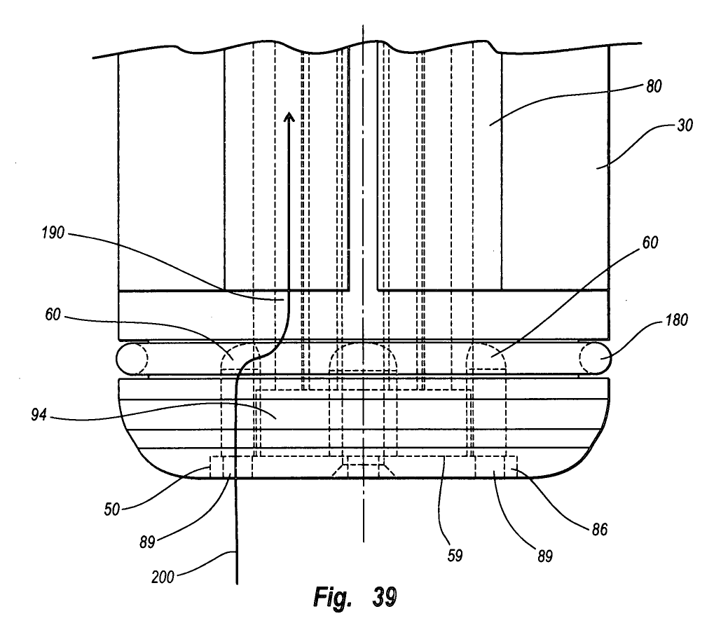

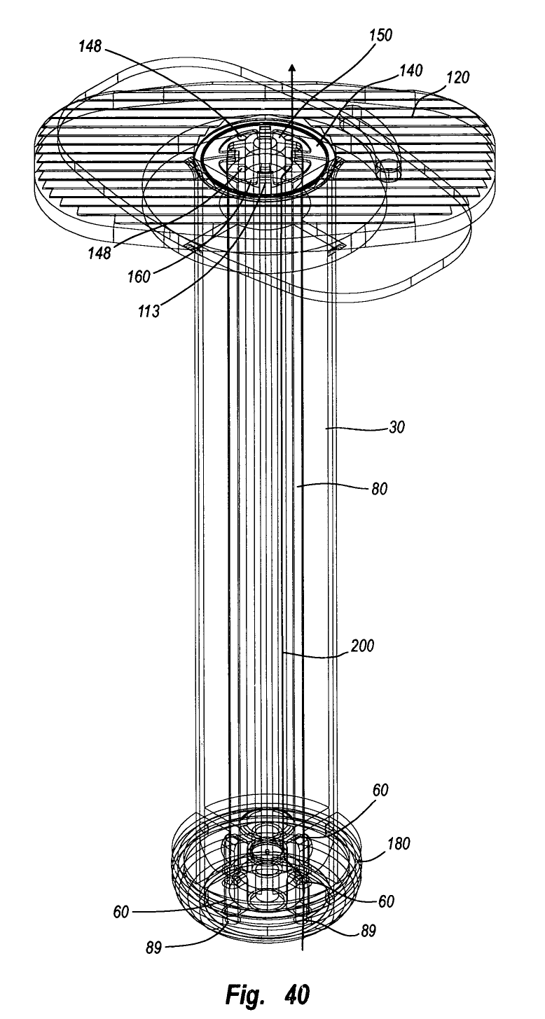

Referring to FIGS. 34-38, piston assembly 6 has inner member 80 rotatably assembled within cannulation 59 of outer member 30 such that the distal surface of distal flange 86 is flush with distal-most surface 46 of outer member 30. Proximal end 84 of inner member 80 protrudes through opening 128 of rotational cap 120. Inner member 80 is retained within outer member 30 by retainer 140 which engages groove 118 of inner member 80. Retainer 140 produces a tensile force in member 80 such that distal flange 86 is held tightly against proximal planar surface 59 of pocket 50 of outer member 80. Seal 160 is retained between the shoulder formed by the intersection of cylindrical pocket 113 with the distal end of cylindrical pocket 110, and seal retainer 150 which is inserted into cylindrical pocket 110. O-ring 180 is installed in groove 47 of outer member 30. Protrusion 37 of outer member 30 engages radial slot 130 of rotational cap 120 so as to limit rotation to no more than angle 133.

Referring to FIG. 39, with inner member rotated as shown in FIGS. 34-38 (“aspirate” position), holes 89 in distal flange 86 of inner member 80 are aligned with grooves 60 in cannulatiion 59 of outer member 30. Grooves 60 allow aspiration flow past cylindrical segment 94 of inner member 80 into axial pockets 190 of inner member 80 to produce aspiration path 200. The aspiration path continues from pockets 190 through holes 135 in rotational cap 120 and through holes 148 in retainer 140.

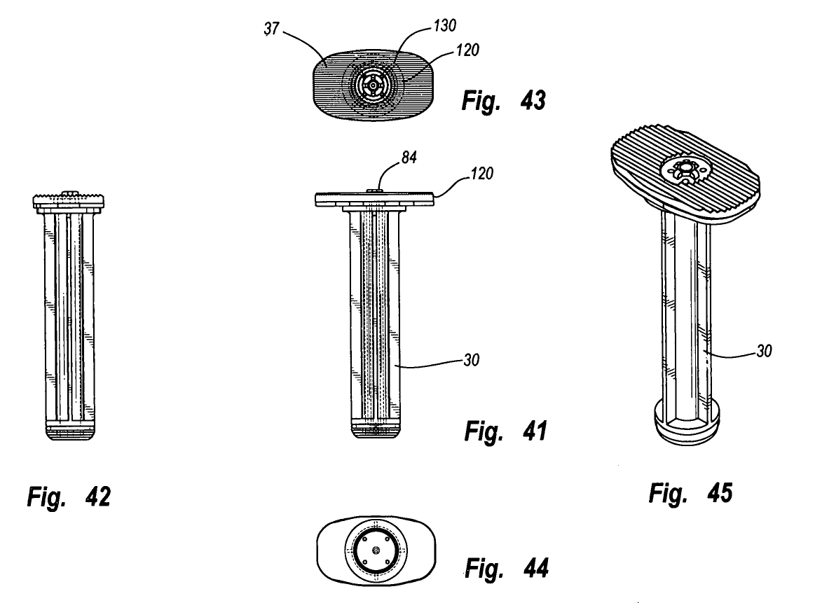

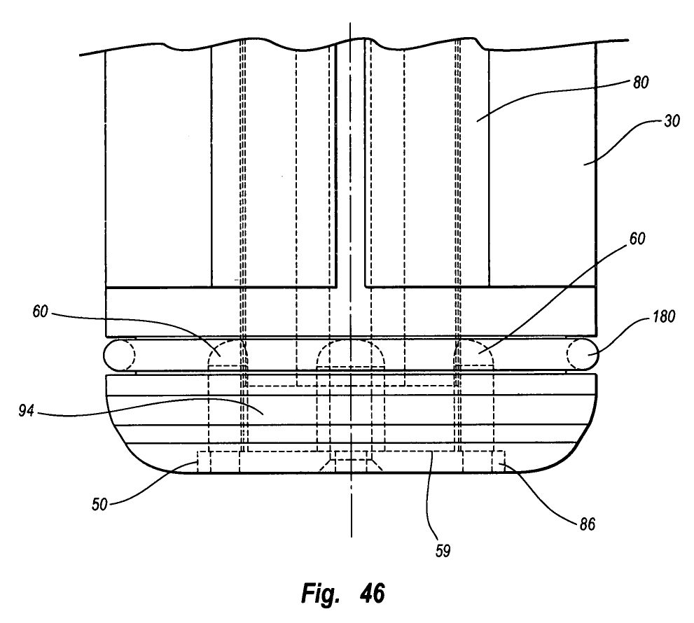

Referring to FIGS. 41-45, with inner member rotated to the “ready” position, rotation is limited by protrusion 37 of outer member 30 acting with radial slot 130 of rotational cap 120. Referring to FIG. 46, with the inner member in the “ready” position, distal flange 86 of inner member 80 prevents flow from entering grooves 60 in cannulation 59 of outer member 30.

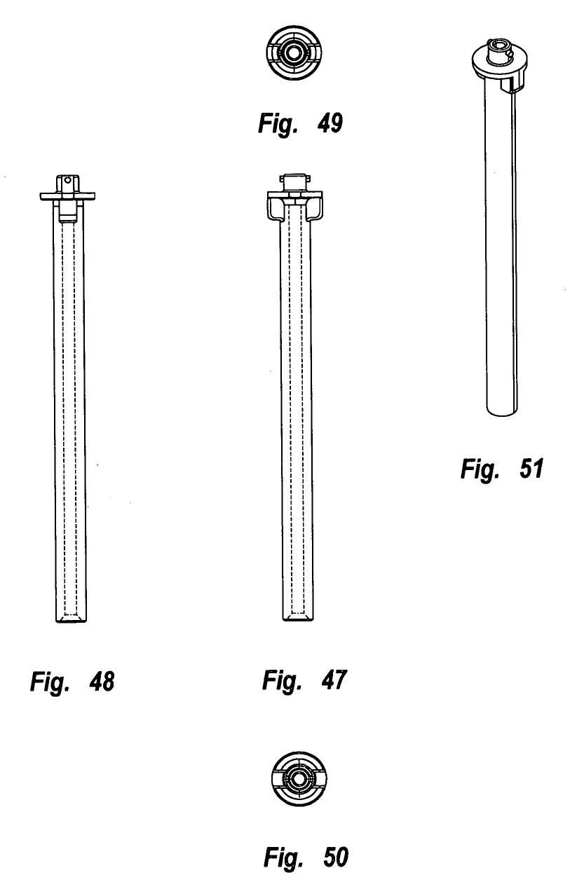

Referring to FIGS. 47-51, nozzle 4 has a proximal end 210 and a distal end 211. Proximal end 210 has a cylindrical portion 212, cylindrical protrusions 213 orthogonal to axis 214 of nozzle 4, and hub 215 which in combination form a J-lock 222. Cylindrical distal region 216 has diameter 217 and length 218. Multiple nozzles having a range of diameters 217 and lengths 218 will be supplied with each instrument. Diameters 217 and length 218 of a nozzle selected for use are determined by the application to which cannulated syringe 1 will be aplied. For instance, some procedures may require a nozzle having a large diameter and short length, while others require a small diameter and long length, or some other combination of diameter 217 and length 218. A cannulation 219 of diameter 220 extends the length of nozzle 4 coaxial with axis 214.

Referring again to FIGS. 1-4, nozzle 4 is mounted to body 5 by J-lock 222.

During use a guide wire is placed to aid in positioning the syringe. Piston assembly 6 is removed from the syringe and desired material to be injected is loaded into body 5. Rotatable cap 120 of piston assembly 6 is rotated to the “aspirate” position. While blocking cannulation 19 of body 5 to prevent loss of material, piston assembly 6 is inserted into body 5 and is advanced until the piston assembly distal end contacts the upper surface of the material and all air is aspirated. Rotatable cap 120 is rotated to the “ready” position. An appropriate nozzle 4 is selected and mounted to syringe 1. Syringe 1 with nozzle 4 is positioned and advanced such that the guide wire passes through cannulation 219 of nozzle 4, through cannulation 81 of inner member 80, through seal 160 which deforms to accept the guide wire diameter, and through seal retainer 150 to exit the syringe proximal end. The syringe is advanced along the guide wire until properly positioned at the desired site. The piston is advanced in the syringe so as to deposit the desired amount of material at the site.

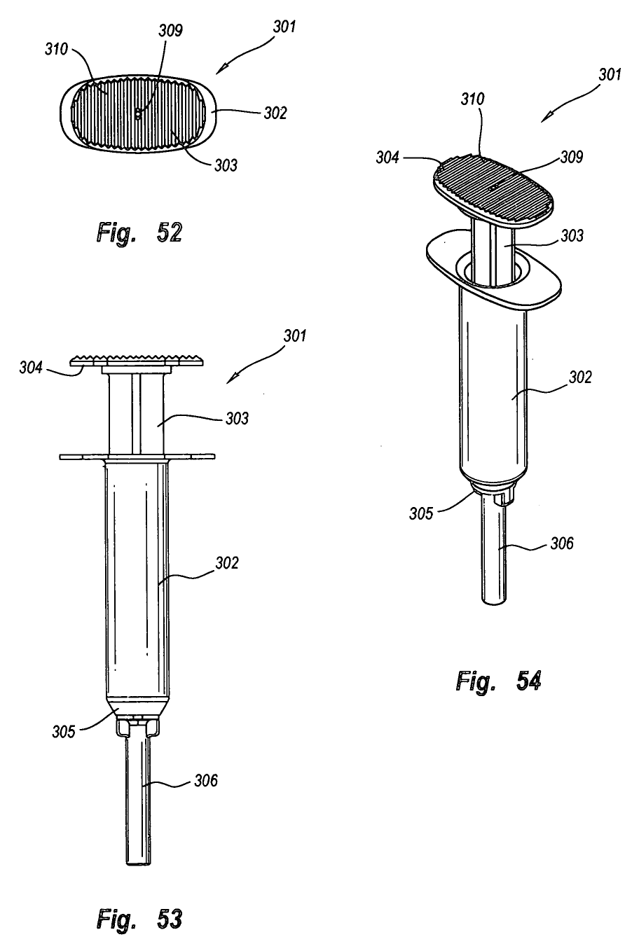

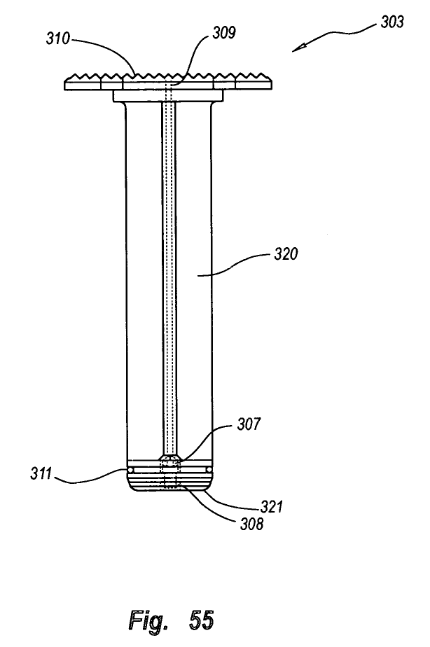

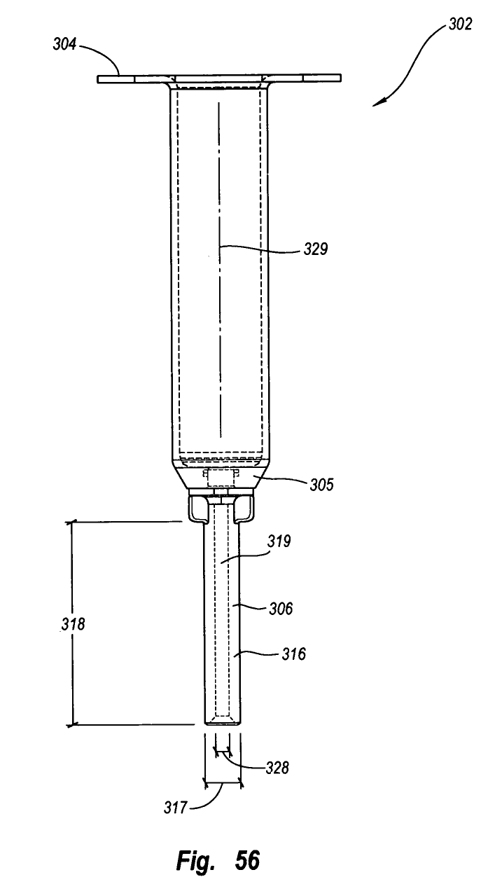

When low viscosity materials which can be sucked into the syringe through the needle are used, or when the material is pre-loaded into the syringe by the manufacturer, it is not necessary for a user surgeon to vent air trapped between the face of the plunger and the material. In the case of low-viscosity materials, the syringe can be positioned with the needle pointed upward and the plunger advanced to expel the air. When the material is supplied pre-loaded in the syringe the air will have bee4n vented prior to shipping to the surgeon. An alternate embodiment of the invention, shown in FIGS. 52-55, has a simplified plunger which does not have a means for venting, but does have a passage to allow the guide wire to pass through the plunger and a sealing means to prevent leakage around the guide wire.

Cannulated injection system 301 has a body 302 and a piston assembly 303, body 302 having a proximal end 304 and a distal end 305 to which is removably mounted nozzle 306, the nozzle being mounted in the same manner as in the previous embodiment. Piston assembly 303 has an elongated portion 320 having seal 307 and retainer 308 mounted in its distal end 321, the seal and the manner of mounting being the same as in the previous embodiment. Cannulation 309 extends axially from seal 307 to proximal end 310 of component 320, cannulation 309 being of a diameter sufficient to allow passage of a guide wire therethrough. Sealing means 311 is mounted to component 320 at its distal end 321.

Referring to nozzle 306, cylindrical distal region 316 has diameter 317 and length 318. Multiple nozzles having a range of diameters 317 and lengths 318 will be supplied with each instrument. Diameter 317 and length 318 of a nozzle selected for use are determined by the application to which cannulated syringe 301 will be applied. For instance, some procedures may require a nozzle having a large diameter and a short length, while other require a small diameter and long length, or some other combination of diameter 317 and length 318. A cannulation 319 of diameter 328 extends the length of nozzle 306 coaxial with axis 329.

During use, a guide wire is placed to aid in positioning syringe 301. A suitable nozzle 306 is selected and mounted to body 302. If the material to be injected is not pre-loaded into syringe 301, material is drawn into syringe 301 through nozzle 306 by partially withdrawing piston assembly 303. Nozzle 306 is then pointed upward and entrapped air expelled by advancing piston assembly 303. Syringe 301 with nozzle 306 is positioned and advanced such that the guide wire passes through cannulation 319 of nozzle 306, through seal retainer 308, through seal 307 whoch deforms to accept the guide wire diameter and through cannulation 309 to exit piston assembly 320 at its proximal end 310. The syringe is advanced along the guide wire until properly positioned at the desired site. The piston is advanced in the syringe so as to deposit the desired amount of injectable material at the site.

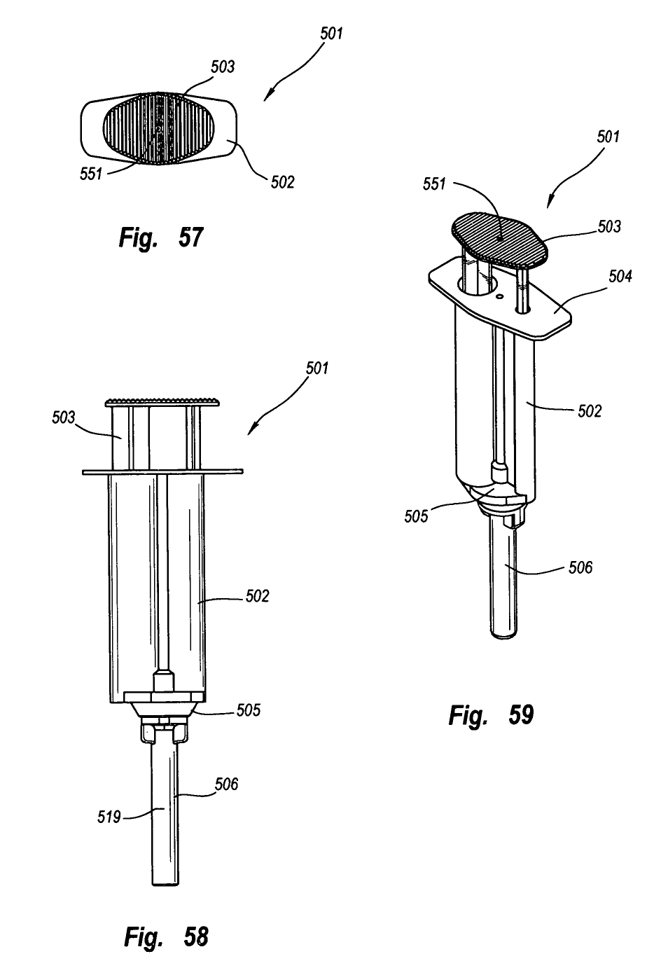

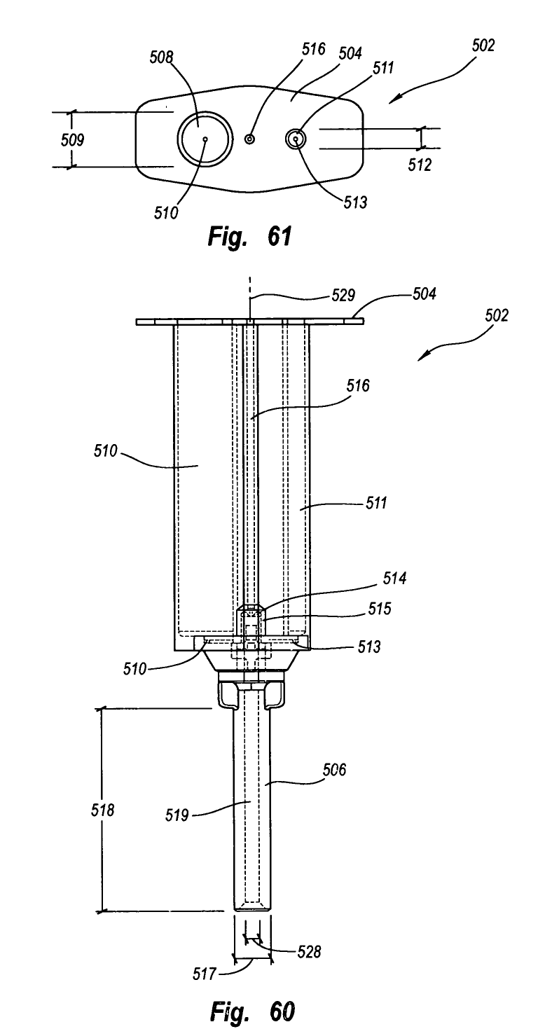

Some materials to be injected are mixed from two components immediately prior to use in a ratio specified by the material manufacturer. Such materials are most efficiently supplied pre-loaded into a syringe which has two barrels, the cross-sectional area of the barrels being of the same ratio as the required mixing ratio of the material. The pistons for the two barrels are mechanically linked so that advancing the pistons causes material to be expelled from each barrel of the syringe simultaneously. An embodiment of the cannulated injection system herein disclosed which is suitable for the injection of two-component materials is shown in FIGS. 57-61. Because the component materials are of low viscosity, venting of trapped air through the piston assembly is not required.

Referring to FIGS. 57-62, cannulated injection system 501 has a body 502 and a piston assembly 503, body 502 having a proximal end 504 and a distal end 505 to which is removably mounted nozzle 506, the nozzle being mounted in the same manner as in the previous embodiments. Body 502 has a first longitudinal cylindrical bore 508 of diameter 509 having a passage 510 at its distal end. Passage 510 allows fluid flow between bore 508 and the proximal end of nozzle 506. A second longitudinal cylindrical bore 511 of diameter 512 has a passage 513 at its distal end. Passage 513 allows fluid flow between bore 511 and the proximal end of nozzle 506. Diameters 509 and 512 are selected such that the ratio of the cross-sectional area of bore 508 to bore 511 is the same as the required mixing ration of the material components. Coaxial with nozzle 506, seal 514 and seal retainer 515 are mounted within body 502 in the same manner as in the previous embodiments. Coaxial with nozzle 506, cannulation 516 having a diameter sufficient for passage of a guide wire therethrough extends from seal 514 to proximal end 504 of body 502.

Referring to nozzle 506, cylindrical distal region 516 has diameter 517 and length 518. Multiple nozzles having a range of diameters 517 and lengths 518 will be supplied with each instrument. Diameter 517 and length 518 of a nozzle selected for use are determined by the application to which cannulated syringe 501 will be applied. For instance, some procedures may require a nozzle having a large diameter and a short length, while other require a small diameter and long length, or some other combination of diameter 517 and length 518. A cannulation 519 of diameter 528 extends the length nozzle 506, coaxial with axis 529.

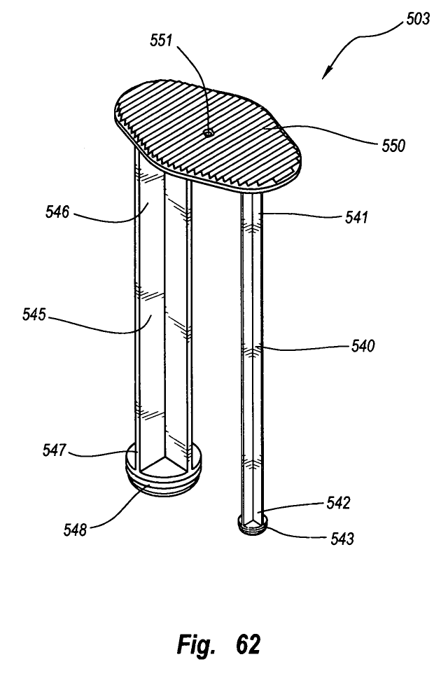

As best seen in FIG. 62, piston assembly 503 has a first elongated portion 540 with a proximal end 541 and a distal end 542 on which is mounted a sealing means 543. Assembly 503 also has a second elongated portion 545 with a proximal end 546 and a distal end 547 on which is mounted sealing means 548. Proximal ends 541 and 546 are joined by a proximal flange 550 having a cylindrical opening 551 positioned so that when piston assembly 503 is assembled to body 502, opening 551 is coaxial with axis 529.

During use, a guide wire is placed to aid in positioning syringe 501. A suitable nozzle 506 is selected and mounted to body 502. Nozzle 506 is then pointed upward and entrapped air is expelled by advancing piston assembly 503. Syringe 501 with nozzle 506 is positioned and advanced such that the guide wire passes through cannulation 519 of nozzle 506; through seal retainer 515; through seal 514, which deforms to accept the guide wire diameter; and through cannulation 516 to exit body 502 at its proximal end 504. The syringe is advanced along the guide wire until properly positioned at the desired site. The piston is advanced in the syringe so as to deposit the desired amount of material at the site.

Although preferred forms of my invention have been herein disclosed, it is to be understood that the present disclosure is by way of example and that variations are possible without departing from the subject matter coming within the scope of the following claims, which subject matter I regard as my invention.

We claim:

1. A cannulated injection system comprising
a hollow body having a proximal end and a distal end;
a piston in said body;
a plunger fixed to said piston and projecting from said hollow body;
an injection tip fixed at and projecting beyond said distal end of said hollow body;
said injection tip having a receiving opening to receive material from the hollow body therethrough and to accommodate passage of a guide wire therethrough; and
means at the proximal end of said hollow body to accommodate passage of the guide wire therethrough, whereby movement of said piston from adjacent the proximal end of said hollow body towards said distal end of said hollow body will eject material from within said hollow body and between said distal end of said hollow body and said piston from said tip.

2. A cannulated injection system as in claim 1, further including
seal means between said piston and said hollow body.

3. A cannulated injection system as in claim 2, further including
a cap on the proximal end of the hollow body; and wherein
the plunger projects through said cap; and
an air passage is provided through said cap.

4. A cannulated injection system as in claim 3, further including
air passage means in the hollow body and extending from the distal side of the piston to the cap; and
means for aligning the air passage means with the air passage through the cap.

5. A cannulated injection system as in claim 1, further including
means for releasably securing an injection tip having a desired passage size therethrough to receive material forced from the hollow body by movement of the piston out the passage through said tip.

6. A cannulated injection system as in claim 1, including
a plurality of hollow bodies;
a piston in each hollow body;
a plunger affixed to and projecting from each piston at the proximal end of a hollow body; and
means connecting proximal ends of the plungers, whereby said plungers move together to move the pistons to which the plungers are connected in unison and to eject material from the distal ends of the hollow bodies through the passageway through the tip.

7. A cannulated injection system as in claim 1, further including
at least one air passage from the interior of the hollow body at the distal end thereof to the hollow body at the proximal end thereof.

8. A cannulated injection system as in claim 7, further including
a rotational cap on the proximal end of the hollow body and an air passageway through the cap to be rotated into alignment with the air passage connected to the distal end of the hollow body.

9. A cannulated injection system as in claim 8, wherein
the means connecting the plungers to move in unison has a guide wire hole therethrough, said guide wire hole being aligned with the passageway through the tip.

10. A cannulated injection system as in claim 2, wherein
the projection tip is axially aligned with the hollow body; and
the means accommodating passage of the guide wire comprises a passage through the proximal end of said hollow body.

11. A cannulated injection system as in claim 10, wherein
the means accommodating passage of the guide wire further comprises a passage through the piston.

12. A cannulated rejection system as in claim 11, further including
seal means in the passage through the piston to sealingly engage a guide wire inserted therethrough.

13. A cannulated rejection system as in claim 12, further including
seal means in the passage through the pistons to sealingly engage a guide wire inserted therethrough.

14. A cannulated injection system as in claim 13, further including
a cap on the proximal end of the hollow body; and wherein
the plunger projects through said cap; and
an air passage is provided through said cap.

15. A cannulated injection system as in claim 14, wherein
the cap has a passage therethrough to accommodate a guide wire passed therethrough.

16. A cannulated injection system as in claim 15, further including
seal means in the passage through the cap to sealingly engage a guide wire passed therethrough.

17. A cannulated injection system as in claim 16, further including
air passage means in the hollow body and extending from the distal side of the piston to the cap; and
means for aligning the air passage means with the air passage through the cap.

18. A cannulated injection system as in claim 17, further including
means for releasably securing an injection tip having a desired passage size therethrough to receive material forced from the hollow body by movement of the piston out the passage through said tip.
